(19) United States
(12) Patent Application Publication
(10) Pub. No.: US 2017/0165179 A1
(43) Pub. Date: Jun. 15, 2017

(54) AGENTS AND METHOD FOR THE TEMPORARY SHAPING OF KERATIN-CONTAINING FIBERS

(71) Applicant: Henkel AG & Co. KGaA, Duesseldorf (DE)

(72) Inventors: Thorsten Knappe, Schenefeld (DE); Pamela Kaftan, Hamburg (DE); Maria Catalina Bermudez Agudelo, Hamburg (DE); Tim Bethge, Hamburg (DE)

(73) Assignee: Henkel AG & Co. KGaA, Duesseldorf (DE)

(21) Appl. No.: 15/360,301

(22) Filed: Nov. 23, 2016

(30) Foreign Application Priority Data
Dec. 15, 2015 (DE) 10 2015 225 199.3

(51) Int. Cl. A61K 8/81; A61K 8/34; B65D 83/14; A61K 8/31; A61K 8/33; A61Q 5/06; A61K 8/04

(52) U.S. Cl. CPC A61K 8/8158; A61K 8/8152; A61Q 5/06; A61K 8/34; A61K 8/046; A61K 8/31; A61K 8/315; A61K 8/33; B65D 83/752; A61K 2800/594; A61K 2800/87

(57) ABSTRACT

Cosmetic agents and methods for the temporary shaping of keratinic fibers using the cosmetic agents are provided herein. In one embodiment, the cosmetic agent for the temporary shaping of keratinic fibers includes a cosmetic preparation. The cosmetic preparation contains at least one copolymer a1), made up of at least the following monomer units: N-tert-octylacrylamide, acrylic acid, and tert-butylaminoethyl methacrylate. The cosmetic preparation further contains at least one copolymer a2), made up of at least the following monomer units: styrene, and at least one of acrylic acid or methacrylic acid. The weight proportion of copolymers a1) and a2) in terms of the total weight of the cosmetic preparation is about 1.0 to about 10% by weight.

### CROSS-REFERENCE TO RELATED APPLICATION

This application claims priority to German Patent Application No. 10 2015 225 199.3, filed Dec. 15, 2015, which is incorporated herein by reference in its entirety.

### TECHNICAL FIELD

This disclosure relates to a cosmetic composition for hair setting or for the temporary reshaping of keratinic fibers, in particular human hair, wherein the composition contains a combination of two specific copolymers.

### BACKGROUND

The temporary creation of hairstyles for a longer time period up to a number of days normally requires the use of active setting substances. Hair treatment agents used for a temporary shaping of the hair therefore play an important role. Suitable agents for temporary shaping typically contain synthetic polymers and/or waxes as an active setting substance. Agents for supporting the temporary reshaping of keratin-containing fibers can be produced, for example, as a hair spray, hair wax, hair gel, or hair foam.

The most important property of an agent for the temporary reshaping of hair, also called a styling agent hereinafter, is to give the treated fibers the greatest possible hold in the new modelled shape, i.e., a shape imposed on the hair. This is also referred to as a strong hairstyle hold or a high degree of hold of the styling agent. The hairstyle hold is determined substantially by the type and amount of the employed active setting substances, but the other components of the styling agent may also have an effect.

In addition to a high degree of hold, styling agents must satisfy a wide range of further requirements. These can be divided roughly into properties on the hair, properties of the particular formulation, e.g., properties of the sprayed aerosol, and properties related to the handling of the styling agent, particular importance being attached to the properties on the hair. Mention can be made in particular of humidity resistance, low tackiness, and a balanced conditioning effect. Furthermore, a styling agent should be universally usable, if possible, for all hair types and be gentle to the hair and skin.

In order to satisfy the different requirements, many synthetic polymers for use in styling agents were already developed as active setting substances. Said polymers can be divided into cationic, anionic, nonionic, and amphoteric setting polymers.

Known amphoteric polymers, used in hair setting products, are based on the monomers, N-tert-octylacrylamide, acrylic acid, and tert-butylaminoethyl methacrylate. Suitable polymers and their use in hair styling gels are described, for example, in the international application WO 2011/012464 A2.

Hair-setting agents based on copolymers of styrene with (meth)acrylic acid and/or esters thereof are described in the international patent application WO2012/168035 A1.

Not every polymer or not every polymer mixture is basically suitable for the production of hair styling agents. This applies in particular to hairsprays in which, for example, the viscosity and thereby also the spraying behavior are influenced by the polymer or the amount of polymer employed.

Even if suitable polymers and polymer combinations have been developed for some time for use in the field of temporary hair shaping, the results achieved so far continue to leave room for improvement, particularly in regard to storage stability, applicability, and the degree of hold of these agents. Currently available styling agents in particular can still be improved with respect to the fact that a good combination of the degree of hold and long-lasting hold (high humidity curl retention) is not always sufficiently assured.

### BRIEF SUMMARY

Cosmetic agents and methods for the temporary shaping of keratinic fibers using the cosmetic agents are provided herein. In one embodiment, the cosmetic agent for the temporary shaping of keratinic fibers includes a cosmetic preparation. The cosmetic preparation contains at least one copolymer a1), made up of at least the following monomer units: N-tert-octylacrylamide, acrylic acid, and tert-butylaminoethyl methacrylate. The cosmetic preparation further contains at least one copolymer a2), made up of at least the following monomer units: styrene, and at least one of acrylic acid or methacrylic acid. The weight proportion of copolymers a1) and a2) in terms of the total weight of the cosmetic preparation is about 1.0 to about 10% by weight.

In another embodiment, the method for the temporary shaping of keratinic fibers includes the step of acting on keratinic fibers by a cosmetic agent. The cosmetic agent includes at least one copolymer a1), made up of at least the following monomer units: N-tert-octylacrylamide, acrylic acid, and tert-butylaminoethyl methacrylate. The cosmetic agent further includes at least one copolymer a2), made up of at least the following monomer units: styrene, and at least one of acrylic acid or methacrylic acid. The weight proportion of copolymers a1) and a2) in terms of the total weight of the cosmetic agent is about 1.0 to about 10% by weight. The method further includes the step of temporarily fixing the keratinic fibers into shape.

### DETAILED DESCRIPTION

The following detailed description is merely exemplary in nature and is not intended to limit the invention or the application and uses of the invention. Furthermore, there is not intention to be bound by any theory presented in the preceding background or the following detained description.

It is contemplated herein to provide other suitable polymer combinations, which are notable for good film-forming and/or setting properties and have a very high degree of hold without having to give up flexibility and good humidity resistance, in particular resistance to sweat and water. The polymer combinations should be suitable, moreover, for producing cosmetic compositions with a high chemical and physical stability and be easy to apply.

This was achieved as contemplated herein by a combination two specific copolymers different from one another. The present disclosure provides:

1. A cosmetic agent for the temporary shaping of keratinic fibers, comprising

a) a cosmetic preparation containing
a1) at least one copolymer, made up of at least the following monomer units:
   N-tert-octylacrylamide,
   acrylic acid,
   tert-butylaminoethyl methacrylate,
a2) at least one copolymer, made up of at least the following monomer units:
   styrene
   acrylic acid and/or methacrylic acid,
      wherein the weight proportion of copolymers a1) and a2) in terms of the total weight of the cosmetic preparation is about 1.0 to about 10% by weight.
      2. The cosmetic agent according to point 1, wherein the weight proportion of copolymers a1) and a2) in terms of the total weight of the cosmetic preparation is about 1.5 and about 9.0% by weight and in particular about 2.0 to about 8.0% by weight.
      3. The cosmetic agent according to one of the preceding points, wherein the at least one copolymer a1), based on its total weight, consists of at least 90% by weight, preferably of at least 95% by weight, and in particular of at least 97% by weight of the monomers, N-tert-octylacrylamide, acrylic acid, and tert-butylaminoethyl methacrylate.
      4. The cosmetic agent according to one of the preceding points, wherein copolymer a1) consists of at least 90% by weight, preferably of at least 95% by weight, and in particular of at least 97% by weight of the monomers: N-tert-octylacrylamide, acrylic acid, tert-butylaminoethyl methacrylate, methyl methacrylate, and hydroxypropyl methacrylate.
      5. The cosmetic agent according to one of the preceding points, wherein copolymer a1) has the INCI name Octylacrylamide/Acrylates/Butylaminoethyl Methacrylate Copolymer.
      6. The cosmetic agent according to one of the preceding points, wherein the preparation contains, based on its total weight, about 0.1 to about 9.9% by weight, preferably about 0.5 to about 8.5% by weight, and in particular about 1.0 to about 7.0% by weight of copolymer a1).
      7. The cosmetic agent according to one of the preceding points, wherein the at least one copolymer a2), based on its total weight, consists of at least 90% by weight, preferably of at least 95% by weight, and in particular of at least 97% by weight of the monomers

styrene,

acrylic acid and/or methacrylic acid.

8. The cosmetic agent according to one of the preceding points, wherein the at least one copolymer a2), based on its total weight, consists of at least 90% by weight, preferably of at least 95% by weight, and in particular of at least 97% by weight of the monomers

styrene,

acrylic acid and/or methacrylic acid,

acrylic acid esters and/or methacrylic acid esters.

9. The cosmetic agent according to one of the preceding points, wherein copolymer a2) has the INCI name Styrene/Acrylates Copolymer.
10. The cosmetic agent according to one of the preceding points, wherein the preparation contains, based on its total weight, about 0.1 to about 9.9% by weight, preferably about 0.5 to about 8.5% by weight, and in particular about 1.0 to about 7.0% by weight of copolymer a2).
11. The cosmetic agent according to one of the preceding points, wherein the weight ratio of copolymer a1) to copolymer a2) is from about 1:7 to about 7:1, preferably from about 1:5 to about 5:1, and in particular from about 1:3 to about 3:1.
12. The cosmetic agent according to one of the preceding points, wherein the preparation contains, based on its total weight, about 40 to about 98% by weight, preferably about 60 to about 95% by weight, and in particular about 70 to about 92% by weight of a polar solvent.
13. The cosmetic agent according to one of the preceding points, wherein the preparation consists, based on its total weight, of at least 70% by weight, preferably of at least 80% by weight, and in particular of at least 90% by weight of the copolymers a1) and a2), ethanol, and/or water.
14. The cosmetic agent according to one of the preceding points, wherein the cosmetic preparation, furthermore, comprises at least one thickener, preferably from the group of polymeric organic thickeners.
15. The cosmetic agent according to one of the preceding points, wherein the cosmetic preparation, furthermore, comprises at least one thickener from the group of anionic polymeric organic thickeners.
16. The cosmetic agent according to one of the preceding points, wherein the cosmetic preparation, furthermore, comprises at least one thickener from the group of anionic, polymeric, amphiphilic organic thickeners
17. The cosmetic agent according to one of the preceding points, wherein the cosmetic preparation, furthermore, comprises at least one thickener from the group with the INCI names: Acrylates/Beheneth-25 Methacrylate Copolymer, Acrylates/Ceteth-20 Itaconate Copolymer, Acrylates/Ceteth-20 Methacrylate Copolymer, Acrylates/Laureth-25 Methacrylate Copolymer, Acrylates/Palmeth-20 Acrylate Copolymer, Acrylates/Palmeth-25 Acrylate Copolymer, Acrylates/Palmeth-25 Itaconate Copolymer, and Acrylates/Steareth-50 Acrylate Copolymer.
18. The cosmetic agent according to one of the preceding points, wherein the preparation contains, based on its total weight, about 0.05 to about 8.0% by weight, preferably about 0.1 to about 5.0% by weight of thickeners.
19. The cosmetic agent according to one of the preceding points, wherein the agent comprises, furthermore,
b) at least one propellant.
   20. The cosmetic agent according to one of the preceding points, wherein the agent comprises, furthermore,
b) at least one propellant from the group comprising propane, a mixture of propane and butane, dimethyl ether, and 1,1-difluoroethane.
   21. The cosmetic agent according to one of the preceding points, comprising, based on its total weight,
a) 30 to 70% by weight of the cosmetic preparation,
b) 30 to 70% by weight of propellant.
   22. A cosmetic product comprising
i) a cosmetic agent according to one of points 1 to 21,
ii) a dispensing device with a spray valve.
   23. Use of an agent or a product according to one of points 1 to 22 for the temporary shaping of keratin-containing fibers, in particular human hair.
   24. A method for the temporary shaping of keratin-containing fibers, in particular human hair, in which the keratinic fibers are acted upon by a cosmetic agent according to one of points 1 to 22 and are temporarily fixed in their shape.

It was found surprisingly in the context of the present disclosure that an improved humidity resistance of styling products can be obtained by the combination of two components known per se, which are already used in styling products. Other usually required properties of styling products such as long-lasting hold, stiffness, and low tackiness are retained or improved in this case. Even if the individual components are known, such a good combination of properties was itself not to be expected and was surprising. It was shown experimentally that by the combination of the two components a highly superadditive, therefore synergistic effect with respect to the humidity resistance and the degree of hold was obtained.

The term ‘keratinic fibers’ as contemplated herein covers pelts, wool, and feathers but in particular human hair.

The essential components of the cosmetic composition of the disclosure are amphoteric copolymer a1) and anionic copolymer a2) different from copolymer a1).

A first essential component of the cosmetic compositions of the disclosure is copolymer a1). In regard to producibility, applicability, and cosmetic effect of the cosmetic agents of the disclosure, it has proven to be advantageous, if the weight proportion of copolymer a1) in terms of the total weight of cosmetic preparation a) is about 0.1 to about 9.9% by weight, preferably about 0.5 to about 8.5% by weight, and in particular of about 1.0 to about 7.0% by weight.

Copolymer a1) is derived from the monomers, N-tert-octylacrylamide, acrylic acid, tert-butylaminoethyl methacrylate, and optionally other monomers.

Preferred copolymers a1) consist preferably of at least 90% by weight, primarily at least 95% by weight, and in particular of at least 97% by weight of the monomers, N-tert-octylacrylamide, acrylic acid, and tert-butylaminoethyl methacrylate. Copolymers a1) are preferably obtained with the exclusive utilization of the monomers, N-tert-octylacrylamide, acrylic acid, and tert-butylaminoethyl methacrylate.

Particularly preferred are copolymers a1) from the monomers: N-tert-octylacrylamide, acrylic acid, tert-butylaminoethyl methacrylate, methyl methacrylate, and hydroxypropyl methacrylate. It is particularly preferable if copolymer a1) consists of at least 90% by weight, preferably of at least 95% by weight, and in particular of at least 97% by weight of the monomers: N-tert-octylacrylamide, acrylic acid, tert-butylaminoethyl methacrylate, methyl methacrylate, and hydroxypropyl methacrylate.

The copolymers a1) described above are marketed, for example, under the name Amphomer® (INCI name: Octylacrylamide/Acrylates/Butylaminoethyl Methacrylate Copolymer; CAS number 70801-07-9) by the company National Starch.

The cosmetic preparations of the disclosure contain an anionic copolymer a2) as a second essential component.

In regard to producibility, applicability, and cosmetic effect of cosmetic agents of the disclosure, it has proven to be advantageous, if the weight proportion of copolymer a2) in terms of the total weight of cosmetic preparation a) is about 0.1 to about 9.9% by weight, preferably about 0.5 to about 8.5% by weight, and in particular of about 1.0 to about 7.0% by weight.

Copolymer a2) is derived from the monomers i) styrene and ii) acrylic acid and/or methacrylic acid, and optionally other monomers.

Preferred copolymers a2) consist preferably of at least 90% by weight, preferably at least 95% by weight, and in particular of at least 97% by weight of the monomers: styrene and acrylic acid and/or methacrylic acid. Particularly preferred copolymers a) were obtained exclusively from the monomers, styrene and acrylic acid and/or methacrylic acid.

The cosmetic agents of a further preferred embodiment are characterized in that the at least one copolymer a2), based on its total weight, consists of at least 90% by weight, preferably of at least 95% by weight, and in particular of at least 97% by weight of the monomers

styrene,

acrylic acid and/or methacrylic acid,

acrylic acid esters and/or methacrylic acid esters.

The copolymers a2) described above are marketed, for example, under the name Acudyne® Shine (INCI name: Styrene/Acrylates Copolymer; CAS number 9010-92-8) by Dow Chemicals.

Copolymer a2) is used in the cosmetic preparation preferably in a partially neutralized or neutralized form. Preferably at least one alkanolamine is used for neutralization. The alkanolamines that can be used as alkalizing agents are preferably selected from primary amines having a C₂-C₆ alkyl parent substance, which carries at least one hydroxyl group. Especially preferred alkanolamines are selected from the group, formed by 2-aminoethan-1-ol (monoethanolamine), tris(2-hydroxyethyl)amine (triethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropane-1,2-diol, and 2-amino-2-methylpropane-1,3-diol. Alkanolamines very especially preferred as contemplated herein are selected from the group of 2-aminoethan-1-ol, 2-amino-2-methylpropan-1-ol, and 2-amino-2-methylpropane-1,3-diol. 2-Amino-2-methylpropanol has proven to be an especially suitable neutralizing agent. Cosmetic agents preferred as contemplated herein contain at least one alkanolamine, preferably 2-amino-2-methylpropanol. 2-Amino-2-methylpropanol is employed in the cosmetic preparations of the disclosure preferably in an amount that does not exceed the amount necessary for the neutralization of copolymer a2). Preferably, the amounts, used in the cosmetic preparations of the disclosure, of 2-amino-2-methylpropanol are about 80 to 100%, especially preferably about 90 to 100%, and especially about 95 to 100% of the amount necessary for the complete neutralization of copolymer a2). In a preferred embodiment, the weight proportion of 2-amino-2-methylpropanol in terms of the total weight of cosmetic agent a) is about 0.1 to about 4.0% by weight, preferably about 0.5 to about 3.0% by weight, and especially about 1.0 to about 2.0% by weight.

The weight proportion of copolymers a1) and a2) in terms of the total weight of the cosmetic preparation is about 1.0 to about 10% by weight. Cosmetic preparations are preferred in which the weight proportion of copolymers a1) and a2) in terms of the total weight of the cosmetic preparation is about 1.5 and about 9.0% by weight and in particular about 2.0 to about 8.0% by weight.

Apart from the total weight proportion of copolymers a1) and a2), furthermore, the weight ratio of copolymers a1) and a2) to one another as well has an effect on the humidity resistance, degree of hold, and the other application properties of the cosmetic agents of the disclosure. Technically especially advantageous cosmetic agents are characterized in that the weight ratio of copolymer a1) to copolymer a2) is from about 1:7 to about 7:1, preferably from about 1:5 to about 5:1, and in particular from about 1:3 to about 3:1.

Apart from the copolymers a1) and copolymers a2) described above, the cosmetic preparations of the disclosure can contain further active substances, auxiliary substances, and care substances.

A first group of active substances employed with preference are the film-forming polymers. These film-forming polymers in this case are not identical to the copolymer a1) or copolymer a2) described above. The weight proportion of the film-forming polymer in terms of the total weight of the cosmetic preparation is preferably about 0.1 to about 8.0% by weight, preferably about 0.5 to about 6.0% by weight, and in particular about 1.0 to about 4.0% by weight.

Nonionic polymers are used with particular preference as film-forming polymers. Suitable nonionic polymers are, for example:
vinylpyrrolidone/vinyl ester copolymers, as they are marketed, for example, under the trademark Luviskol® (BASF). Luviskol® VA 64 and Luviskol® VA 73, each being vinylpyrrolidone/vinyl acetate copolymers, are preferred nonionic polymers.
cellulose ethers, such as hydroxypropyl cellulose, hydroxyethyl cellulose, and methyl hydroxypropyl cellulose, as they are marketed, for example, under the trademarks Culminal® and Benecel® (AQUALON).
shellac.
polyvinylpyrrolidones, as they are marketed, for example, under the name Luviskol® (BASF).
siloxanes. These siloxanes can be both water-soluble and water-insoluble. Both volatile and nonvolatile siloxanes are suitable, nonvolatile siloxanes being understood to be compounds whose boiling point at normal pressure is above 200° C. Preferred siloxanes are polydialkylsiloxanes such as, for example, polydimethylsiloxane, polyalkylarylsiloxanes such as, for example, polyphenylmethylsiloxane, ethoxylated polydialkylsiloxanes, and polydialkylsiloxanes that contain amine and/or hydroxy groups.
glycosidically substituted silicones.

Because of their cosmetic effect in combination with copolymers a1) and a2), film-forming polymers preferably employed as contemplated herein are in particular the polyvinylpyrrolidones (INCI name: PVP) and the vinylpyrrolidone/vinyl acetate copolymers (INCI name VP/VA Copolymer), the weight proportion of these polymers preferably being limited to amounts between about 1.0 and about 10% by weight. Particularly preferred cosmetic preparations of the disclosure are therefore characterized in that they contain, based on their total weight, further about 1.0 to about 10% by weight of polyvinylpyrrolidone and/or vinylpyrrolidone/vinyl acetate copolymer, preferably polyvinylpyrrolidone. Particularly preferred cosmetic preparations have a weight proportion of the polyvinylpyrrolidone and/or vinylpyrrolidone/vinyl acetate copolymer in terms of the total weight of the cosmetic preparation of about 2.0 to about 8.5% by weight, preferably of about 3.0 to about 7.0% by weight.

In summary, cosmetic agents particularly preferred as contemplated herein with copolymers a1) and a2) and film-forming polymer a3) contain three polymers different from one another.

Protein hydrolysates and/or derivatives thereof can be used as care substances. Protein hydrolysates are product mixtures obtained by acid-, base-, or enzyme-catalyzed degradation of proteins. The term ‘protein hydrolysates’ as contemplated herein is also understood to be total hydrolysates, as well as individual amino acids and derivatives thereof, and mixtures of different amino acids. The molar weight of protein hydrolysates usable as contemplated herein is between about 75 (the molar weight of glycine) and about 200,000; the molar weight is preferably about 75 to about 50,000 Da, and very particularly preferably about 75 to about 20,000 Da.

A further group of care substances are vitamins, provitamins, vitamin precursors, and/or derivatives thereof. In this case, the vitamins, provitamins, and vitamin precursors that are usually assigned to the groups A, B, C, E, F, and H are preferred as contemplated herein.

Other care substances are glycerol, propylene glycol, panthenol, caffeine, nicotinamide, and sorbitol.

Plant extracts, but also mono- or oligosaccharides and/or lipids can be used as care substances.

The composition of some cosmetic preparations a), in which the weight proportion of copolymers a1) and a2) in terms of the total weight of the cosmetic preparation is about 1.0 to about 10% by weight, preferably about 1.5 to about 9.0% by weight, and in particular about 2.0 to about 8.0% by weight, can be obtained from the following tables. (The quantities in percent by weight refer to the total weight of the cosmetic agent unless specified otherwise.)

| | Formula 1 | Formula 2 | Formula 3 | Formula 4 | Formula 5 |
|---|---|---|---|---|---|
| Copolymer a1)* | 0.1 to 9.9 | 0.1 to 8.9 | 0.5 to 8.0 | 0.5 to 7.5 | 1.0 to 7.0 |
| Copolymer a2)* | 0.1 to 9.9 | 0.1 to 8.9 | 0.5 to 8.0 | 0.5 to 7.5 | 1.0 to 7.0 |
| Optional additives | To 100 | To 100 | To 100 | To 100 | To 100 |
| | Formula 6 | Formula 7 | Formula 8 | Formula 9 | Formula 10 |
| Octylacrylamide/ | 0.1 to 9.9 | 0.1 to 8.9 | 0.5 to 8.0 | 0.5 to 7.5 | 1.0 to 7.0 |
| Acrylates/ | | | | | |
| utylaminoethyl | | | | | |
| Methacrylate | | | | | |
| Copolymer (INCI) | | | | | |
| Copolymer a2)* | 0.1 to 9.9 | 0.1 to 8.9 | 0.5 to 8.0 | 0.5 to 7.5 | 1.0 to 7.0 |
| Optional additives | To 100 | To 100 | To 100 | To 100 | To 100 |
| | Formula 11 | Formula 12 | Formula 13 | Formula 14 | Formula 15 |
| Copolymer a1)* | 0.1 to 9.9 | 0.1 to 8.9 | 0.5 to 8.0 | 0.5 to 7.5 | 1.0 to 7.0 |
| Styrene/Acrylates | 0.1 to 9.9 | 0.1 to 8.9 | 0.5 to 8.0 | 0.5 to 7.5 | 1.0 to 7.0 |
| Copolymer (INCI) | | | | | |
| Optional additives | To 100 | To 100 | To 100 | To 100 | To 100 |
| | Formula 16 | Formula 17 | Formula 18 | Formula 19 | Formula 20 |
| Octylacrylamide/ | 0.1 to 9.9 | 0.1 to 8.9 | 0.5 to 8.0 | 0.5 to 7.5 | 1.0 to 7.0 |
| Acrylates/ | | | | | |
| utylaminoethyl | | | | | |
| ethacrylate | | | | | |
| Copolymer (INCI) | | | | | |
| Styrene/Acrylates | 0.1 to 9.9 | 0.1 to 8.9 | 0.5 to 8.0 | 0.5 to 7.5 | 1.0 to 7.0 |
| Copolymer (INCI) | | | | | |
| Optional additives | To 100 | To 100 | To 100 | To 100 | To 100 |

*according to claim 1

The cosmetic preparations of the disclosure can be produced in different ways. In this case, the weight proportion and the precise composition of the liquid carrier optionally present in the cosmetic preparation have an essential effect on the final production form of these preparations.

Preferred cosmetic agents are based on an aqueous, aqueous/alcoholic, or alcoholic carrier. Thus, preferred cosmetic agents, based on their total weight, contain about 40 to about 98% by weight, preferably about 60 to about 95% by weight, and in particular about 70 to about 92% by weight of a polar solvent, preferably a polar solvent from the group comprising water, ethanol, and isopropanol.

As already mentioned, in particular the lower alcohols, usually used for cosmetic purposes and having 1 to 4 carbon atoms, such as, for example, ethanol and isopropanol, can be contained as alcohols.

Apart from these alcoholic solvents, furthermore, water-soluble cosolvents can be used in particular in combination with water. Examples of particularly preferred cosolvents are glycerol and/or ethylene glycol and/or 1,2-propylene glycol, which are preferably used in an amount of 0 to 30% by weight based on cosmetic preparation a).

Together with the other above-described copolymers a1) and a2), the aqueous, aqueous/alcoholic, or alcoholic carriers preferably form an essential component of cosmetic preparations a) of the disclosure. Particularly preferred are cosmetic preparations that consist, based on their total weight, of at least 70% by weight, preferably of at least 80% by weight, and in particular of at least 90% by weight of copolymers a1) and a2), ethanol, and/or water.

The composition of some technically advantageous cosmetic preparations a) with a liquid carrier, in which the weight proportion of copolymers a1) and a2), in terms of the total weight of the cosmetic preparation, is about 1.0 to about 10% by weight, preferably about 1.5 to about 9.0% by weight, and in particular about 2.0 to about 8.0% by weight, can be obtained from the following tables. (The quantities in percent by weight refer to the total weight of the cosmetic agent unless specified otherwise.)

| | Formula 21 | Formula 22 | Formula 23 | Formula 24 | Formula 25 |
|---|---|---|---|---|---|
| Copolymer a1)* | 0.1 to 9.9 | 0.1 to 8.9 | 0.5 to 8.0 | 0.5 to 7.5 | 1.0 to 7.0 |
| Copolymer a2)* | 0.1 to 9.9 | 0.1 to 8.9 | 0.5 to 8.0 | 0.5 to 7.5 | 1.0 to 7.0 |
| Water and/or ethanol | 40 to 98 | 40 to 98 | 60 to 95 | 60 to 95 | 70 to 92 |
| Optional additives | To 100 | To 100 | To 100 | To 100 | To 100 |
| | Formula 26 | Formula 27 | Formula 28 | Formula 29 | Formula 30 |
| Octylacrylamide/ | 0.1 to 9.9 | 0.1 to 8.9 | 0.5 to 8.0 | 0.5 to 7.5 | 1.0 to 7.0 |
| Acrylates/ | | | | | |
| Butylaminoethyl | | | | | |
| Methacrylate | | | | | |
| Copolymer (INCI) | | | | | |
| Copolymer a2)* | 0.1 to 9.9 | 0.1 to 8.9 | 0.5 to 8.0 | 0.5 to 7.5 | 1.0 to 7.0 |
| Water and/or ethanol | 40 to 98 | 40 to 98 | 60 to 95 | 60 to 95 | 70 to 92 |
| Optional additives | To 100 | To 100 | To 100 | To 100 | To 100 |
| | Formula 31 | Formula 32 | Formula 33 | Formula 34 | Formula 35 |
| Copolymer a1)* | 0.1 to 9.9 | 0.1 to 8.9 | 0.5 to 8.0 | 0.5 to 7.5 | 1.0 to 7.0 |
| Styrene/Acrylates | 0.1 to 9.9 | 0.1 to 8.9 | 0.5 to 8.0 | 0.5 to 7.5 | 1.0 to 7.0 |
| Copolymer (INCI) | | | | | |
| Water and/or ethanol | 40 to 98 | 40 to 98 | 60 to 95 | 60 to 95 | 70 to 92 |
| Optional additives | To 100 | To 100 | To 100 | To 100 | To 100 |
| | Formula 36 | Formula 37 | Formula 38 | Formula 39 | Formula 40 |
| Octylacrylamide/ | 0.1 to 9.9 | 0.1 to 8.9 | 0.5 to 8.0 | 0.5 to 7.5 | 1.0 to 7.0 |
| Acrylates/ | | | | | |
| Butylaminoethyl | | | | | |
| Methacrylate | | | | | |
| Copolymer (INCI) | | | | | |
| Styrene/Acrylates | 0.1 to 9.9 | 0.1 to 8.9 | 0.5 to 8.0 | 0.5 to 7.5 | 1.0 to 7.0 |
| Copolymer (INCI) | | | | | |
| Water and/or ethanol | 40 to 98 | 40 to 98 | 60 to 95 | 60 to 95 | 70 to 92 |
| Optional additives | To 100 | To 100 | To 100 | To 100 | To 100 |

*according to claim 1

Of course, it is not only the weight proportion of the liquid carrier in terms of the total weight of cosmetic preparation a) that can vary, but the weight ratio of the aqueous to alcoholic carrier is also changeable.

The composition of some technically advantageous cosmetic preparations a) with a liquid carrier, in which the weight proportion of copolymers a1) and a2) in terms of the total weight of the cosmetic preparation is about 1.0 to about 10% by weight, preferably about 1.5 to about 9.0% by weight, and in particular about 2.0 to about 8.0% by weight, can be obtained from the following tables. (The quantities in percent by weight refer to the total weight of the cosmetic agent unless specified otherwise.) Appropriate cosmetic preparations are suitable, for example, as pump sprays.

Cosmetic preparations a) with a high ethanol content

| | Formula 41 | Formula 42 | Formula 43 | Formula 44 | Formula 45 |
|---|---|---|---|---|---|
| Copolymer a1)* | 0.1 to 9.9 | 0.1 to 8.9 | 0.5 to 8.0 | 0.5 to 7.5 | 1.0 to 7.0 |
| Copolymer a2)* | 0.1 to 9.9 | 0.1 to 8.9 | 0.5 to 8.0 | 0.5 to 7.5 | 1.0 to 7.0 |
| Ethanol | 60 to 98 | 60 to 98 | 60 to 98 | 60 to 98 | 60 to 98 |
| Water | 0 to 10 | 0 to 10 | 0 to 10 | 0 to 10 | 0 to 10 |
| Optional additives | To 100 | To 100 | To 100 | To 100 | To 100 |
| | Formula 46 | Formula 47 | Formula 48 | Formula 49 | Formula 50 |
| Octylacrylamide/ | 0.1 to 9.9 | 0.1 to 8.9 | 0.5 to 8.0 | 0.5 to 7.5 | 1.0 to 7.0 |
| Acrylates/ | | | | | |
| Butylaminoethyl | | | | | |
| Methacrylate | | | | | |
| Copolymer (INCI) | | | | | |
| Copolymer a2)* | 0.1 to 9.9 | 0.1 to 8.9 | 0.5 to 8.0 | 0.5 to 7.5 | 1.0 to 7.0 |
| Ethanol | 60 to 98 | 60 to 98 | 60 to 98 | 60 to 98 | 60 to 98 |
| Water | 0 to 10 | 0 to 10 | 0 to 10 | 0 to 10 | 0 to 10 |
| Optional additives | To 100 | To 100 | To 100 | To 100 | To 100 |
| | Formula 51 | Formula 52 | Formula 53 | Formula 54 | Formula 55 |
| Copolymer a1)* | 0.1 to 9.9 | 0.1 to 8.9 | 0.5 to 8.0 | 0.5 to 7.5 | 1.0 to 7.0 |
| Styrene/Acrylates | 0.1 to 9.9 | 0.1 to 8.9 | 0.5 to 8.0 | 0.5 to 7.5 | 1.0 to 7.0 |
| Copolymer (INCI) | | | | | |
| Ethanol | 60 to 98 | 60 to 98 | 60 to 98 | 60 to 98 | 60 to 98 |
| Water | 0 to 10 | 0 to 10 | 0 to 10 | 0 to 10 | 0 to 10 |
| Optional additives | To 100 | To 100 | To 100 | To 100 | To 100 |
| | Formula 56 | Formula 57 | Formula 58 | Formula 59 | Formula 60 |
| Octylacrylamide/ | 0.1 to 9.9 | 0.1 to 8.9 | 0.5 to 8.0 | 0.5 to 7.5 | 1.0 to 7.0 |
| Acrylates/ | | | | | |
| Butylaminoethyl | | | | | |
| Methacrylate | | | | | |
| Copolymer (INCI) | | | | | |
| Styrene/Acrylates | 0.1 to 9.9 | 0.1 to 8.9 | 0.5 to 8.0 | 0.5 to 7.5 | 1.0 to 7.0 |
| Copolymer (INCI) | | | | | |
| Ethanol | 60 to 98 | 60 to 98 | 60 to 98 | 60 to 98 | 60 to 98 |
| Water | 0 to 10 | 0 to 10 | 0 to 10 | 0 to 10 | 0 to 10 |
| Optional additives | To 100 | To 100 | To 100 | To 100 | To 100 |

*according to claim 1

Cosmetic preparations a) with a medium ethanol content

| | Formula 61 | Formula 62 | Formula 63 | Formula 64 | Formula 65 |
|---|---|---|---|---|---|
| Copolymer a1)* | 0.1 to 9.9 | 0.1 to 8.9 | 0.5 to 8.0 | 0.5 to 7.5 | 1.0 to 7.0 |
| Copolymer a2)* | 0.1 to 9.9 | 0.1 to 8.9 | 0.5 to 8.0 | 0.5 to 7.5 | 1.0 to 7.0 |
| Ethanol | 10 to 50 | 15 to 50 | 20 to 50 | 25 to 50 | 30 to 50 |
| Water | 2.0 to 40 | 2.0 to 40 | 2.0 to 40 | 2.0 to 40 | 2.0 to 40 |
| Optional additives | To 100 | To 100 | To 100 | To 100 | To 100 |
| | Formula 66 | Formula 67 | Formula 68 | Formula 69 | Formula 70 |
| Octylacrylamide/ | 0.1 to 9.9 | 0.1 to 8.9 | 0.5 to 8.0 | 0.5 to 7.5 | 1.0 to 7.0 |
| Acrylates/ | | | | | |
| Butylaminoethyl | | | | | |
| Methacrylate | | | | | |
| Copolymer (INCI) | | | | | |
| Copolymer a2)* | 0.1 to 9.9 | 0.1 to 8.9 | 0.5 to 8.0 | 0.5 to 7.5 | 1.0 to 7.0 |
| Ethanol | 10 to 50 | 15 to 50 | 20 to 50 | 25 to 50 | 30 to 50 |
| Water | 2.0 to 40 | 2.0 to 40 | 2.0 to 40 | 2.0 to 40 | 2.0 to 40 |
| Optional additives | To 100 | To 100 | To 100 | To 100 | To 100 |
| | Formula 71 | Formula 72 | Formula 73 | Formula 74 | Formula 75 |
| Copolymer a1)* | 0.1 to 9.9 | 0.1 to 8.9 | 0.5 to 8.0 | 0.5 to 7.5 | 1.0 to 7.0 |
| Styrene/Acrylates | 0.1 to 9.9 | 0.1 to 8.9 | 0.5 to 8.0 | 0.5 to 7.5 | 1.0 to 7.0 |
| Copolymer (INCI) | | | | | |
| Ethanol | 10 to 50 | 15 to 50 | 20 to 50 | 25 to 50 | 30 to 50 |
| Water | 2.0 to 40 | 2.0 to 40 | 2.0 to 40 | 2.0 to 40 | 2.0 to 40 |
| Optional additives | To 100 | To 100 | To 100 | To 100 | To 100 |
| | Formula 76 | Formula 77 | Formula 78 | Formula 79 | Formula 80 |
| Octylacrylamide/ | 0.1 to 9.9 | 0.1 to 8.9 | 0.5 to 8.0 | 0.5 to 7.5 | 1.0 to 7.0 |
| Acrylates/ | | | | | |
| Butylaminoethyl | | | | | |
| Methacrylate | | | | | |
| Copolymer (INCI) | | | | | |
| Styrene/Acrylates | 0.1 to 9.9 | 0.1 to 8.9 | 0.5 to 8.0 | 0.5 to 7.5 | 1.0 to 7.0 |
| Copolymer (INCI) | | | | | |
| Ethanol | 10 to 50 | 15 to 50 | 20 to 50 | 25 to 50 | 30 to 50 |
| Water | 2.0 to 40 | 2.0 to 40 | 2.0 to 40 | 2.0 to 40 | 2.0 to 40 |
| Optional additives | To 100 | To 100 | To 100 | To 100 | To 100 |

*according to claim 1

Cosmetic preparations a) with a low ethanol content

| | Formula 81 | Formula 82 | Formula 83 | Formula 84 | Formula 85 |
|---|---|---|---|---|---|
| Copolymer a1)* | 0.1 to 9.9 | 0.1 to 8.9 | 0.5 to 8.0 | 0.5 to 7.5 | 1.0 to 7.0 |
| Copolymer a2)* | 0.1 to 9.9 | 0.1 to 8.9 | 0.5 to 8.0 | 0.5 to 7.5 | 1.0 to 7.0 |
| Ethanol | 0 to 20 | 0 to 20 | 0 to 20 | 0 to 5.0 | 0 to 5.0 |
| Water | 50 to 97 | 60 to 97 | 60 to 97 | 65 to 97 | 65 to 97 |
| Optional additives | To 100 | To 100 | To 100 | To 100 | To 100 |
| | Formula 86 | Formula 87 | Formula 88 | Formula 89 | Formula 90 |
| Octylacrylamide/ | 0.1 to 9.9 | 0.1 to 8.9 | 0.5 to 8.0 | 0.5 to 7.5 | 1.0 to 7.0 |
| Acrylates/ | | | | | |
| Butylaminoethyl | | | | | |
| Methacrylate | | | | | |
| Copolymer (INCI) | | | | | |
| Copolymer a2)* | 0.1 to 9.9 | 0.1 to 8.9 | 0.5 to 8.0 | 0.5 to 7.5 | 1.0 to 7.0 |
| Ethanol | 0 to 20 | 0 to 20 | 0 to 20 | 0 to 5.0 | 0 to 5.0 |
| Water | 50 to 97 | 60 to 97 | 60 to 97 | 65 to 97 | 65 to 97 |
| Optional additives | To 100 | To 100 | To 100 | To 100 | To 100 |
| | Formula 91 | Formula 92 | Formula 93 | Formula 94 | Formula 95 |
| Copolymer a1)* | 0.1 to 9.9 | 0.1 to 8.9 | 0.5 to 8.0 | 0.5 to 7.5 | 1.0 to 7.0 |
| Styrene/Acrylates | 0.1 to 9.9 | 0.1 to 8.9 | 0.5 to 8.0 | 0.5 to 7.5 | 1.0 to 7.0 |
| Copolymer (INCI) | | | | | |
| Ethanol | 0 to 20 | 0 to 20 | 0 to 20 | 0 to 5.0 | 0 to 5.0 |
| Water | 50 to 97 | 60 to 97 | 60 to 97 | 65 to 97 | 65 to 97 |
| Optional additives | To 100 | To 100 | To 100 | To 100 | To 100 |
| | Formula 96 | Formula 97 | Formula 98 | Formula 99 | Formula 100 |
| Octylacrylamide/ | 0.1 to 9.9 | 0.1 to 8.9 | 0.5 to 8.0 | 0.5 to 7.5 | 1.0 to 7.0 |
| Acrylates/ | | | | | |
| Butylaminoethyl | | | | | |
| Methacrylate | | | | | |
| Copolymer (INCI) | | | | | |
| Styrene/Acrylates | 0.1 to 9.9 | 0.1 to 8.9 | 0.5 to 8.0 | 0.5 to 7.5 | 1.0 to 7.0 |
| Copolymer (INCI) | | | | | |
| Ethanol | 0 to 20 | 0 to 20 | 0 to 20 | 0 to 5.0 | 0 to 5.0 |
| Water | 50 to 97 | 60 to 97 | 60 to 97 | 65 to 97 | 65 to 97 |
| Optional additives | To 100 | To 100 | To 100 | To 100 | To 100 |

*according to claim 1

Conceivable production forms for cosmetic preparations a) of the disclosure are creams and lotions but gels as well. Moreover, these preparations are suitable further, however, also for use as a mousse, foam, or spray.

Gel-like cosmetic preparations contain at least one thickener as a further ingredient. In regard to producibility, applicability, and cosmetic effect of cosmetic preparations of the disclosure, it has proven to be advantageous, if the weight proportion of thickener a2) in terms of the total weight of cosmetic preparation a) is about 0.05 to about 8.0% by weight, preferably about 0.1 to about 5.0% by weight.

Preferred thickeners are selected from the group of polymeric organic thickeners. The polymeric organic thickeners can be crosslinked or non-crosslinked.

Preferred thickeners are selected from the group of polymeric organic thickeners. A first group of particularly preferred thickeners a2) contain at least one structural unit selected from at least one structural unit of the formula (I) or salt forms thereof or at least one structural unit (II) or salt forms thereof,

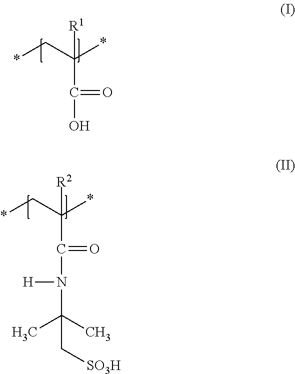

where R¹ and R² independently of one another stand for a hydrogen atom or a methyl group.

According to the above formulas and all of the following formulas, a chemical bond labeled with the symbol * stands for a free valence of the corresponding structural fragment.

Particularly preferred anionic polymers with a thickening action contain at least one structural unit of the formula (I). Acryl acid homopolymers form a first group of particularly preferred thickeners.

Particularly preferred thickeners are
polyacrylic acids with the INCI name Carbomer, as they are marketed, for example, by the company 3V Sigma under the trade name Synthalen® K or by the company Lubrizol under the trade name Carbopol.

The anionic, polymeric, amphiphilic thickeners form a second particularly preferred group of thickeners a2). Suitable thickeners comprise preferably at least one structural unit of the formula (III) and at least one structural unit of the formula (IV),

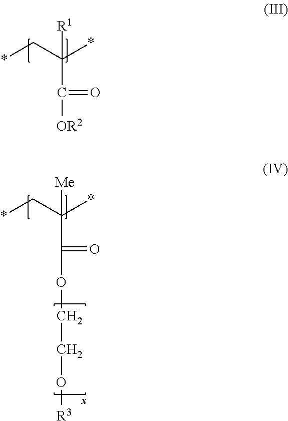

where

R¹ stands for a hydrogen atom or a methyl group,

R² stands for a hydrogen atom or a (C₁ to C₆) alkyl group,

R³ stands for a (C₈ to C₃₀) alkyl group,

M⁺ stands for a physiologically acceptable cation, and

x stands for an integer from 0 to 35.

Preferred in this case are in particular thickeners with the INCI names: Acrylates/Beheneth-25 Methacrylate Copolymer, Acrylates/Ceteth-20 Itaconate Copolymer, Acrylates/Ceteth-20 Methacrylate Copolymer, Acrylates/Laureth-25 Methacrylate Copolymer, Acrylates/Palmeth-20 Acrylate Copolymer, Acrylates/Palmeth-25 Acrylate Copolymer, Acrylates/Palmeth-25 Itaconate Copolymer, and Acrylates/Steareth-50 Acrylate Copolymer.

Particularly preferred thickeners are
thickeners with the INCI name Acrylates/Steareth-20 Methacrylate Copolymer, as they are marketed, for example, under the trade name Aculyn® 22 by the company Rohm & Haas;
thickeners with the INCI name Acrylates/Steareth-20 Methacrylate Crosspolymer, as they are marketed, for example, under the trade name Aculyn® 88 by the company Rohm & Haas;
thickeners with the INCI name Acrylates/Steareth-20 Itaconate Copolymer, as they are marketed, for example, under the trade name Structure 2001 by the company National Starch.

Further anionic, polymeric, amphiphilic thickeners are characterized by long-chain alkyl substituents. This group includes, for example, the compounds with the INCI names Acrylates/Stearyl Methacrylate Copolymer and Acrylates/Vinyl Isodecanoate Crosspolymer.

Particularly preferred thickeners are
thickeners with the INCI name Acrylates/C₁₀₋₃₀ Alkyl Acrylate Crosspolymer, as they are marketed, for example, under the trade name Carbopol Ultrez 21 by the company Lubrizol.

Further thickeners can be selected, for example, from the polymeric thickening agents known under the following INCI names: Acrylamides Copolymer, Acrylamide/Sodium Acrylate Copolymer, Acrylamide/Sodium Acryloyldimethyltaurate Copolymer, Acrylates/Acetoacetoxyethyl Methacrylate Copolymer, Acrylic Acid/Acrylonitrogens Copolymer, Agar, Agarose, Alcaligenes Polysaccharides, Algin, Alginic Acid, Ammonium Acrylates/Acrylonitrogens Copolymer, Ammonium Acrylates Copolymer, Ammonium Acryloyldimethyltaurate/Vinyl Formamide Copolymer, Ammonium Acryloyldimethyltaurate/VP Copolymer, Ammonium Alginate, Ammonium Polyacryloyldimethyl Taurate, Amylopectin, Ascorbyl Methylsilanol Pectinate, Astragalus Gummifer Gum, Attapulgite, Avena Sativa (Oat) Kernel Flour, Bentonite, Butoxy Chitosan, Caesalpinia Spinosa Gum, Calcium Alginate, Calcium Carboxymethyl Cellulose, Calcium Carrageenan, Calcium Potassium Carbomer, Calcium Starch Octenylsuccinate, C20-40 Alkyl Stearate, Carboxybutyl Chitosan, Carboxymethyl Chitin, Carboxymethyl Chitosan, Carboxymethyl Dextran, Carboxymethyl Hydroxyethylcellulose, Carboxymethyl Hydroxypropyl Guar, Cellulose Acetate Propionate Carboxylate, Cellulose Gum, Ceratonia Siliqua Gum, Cetyl Hydroxyethylcellulose, Cholesterol/HDI/Pullulan Copolymer, Cholesteryl Hexyl Dicarbamate Pullulan, Cyamopsis Tetragonoloba (Guar) Gum, Diglycol/CHDM/Isophthalates/SIP Copolymer, Dihydrogenated Tallow Benzylmonium Hectorite, Dimethicone Crosspolymer-2, Dimethicone Propyl PG-Betaine, DMAPA Acrylates/Acrylic Acid/Acrylonitrogens Copolymer, Ethylene/Sodium Acrylate Copolymer, Gelatin, Gellan Gum, Glyceryl Alginate, Glycine Soja (Soybean) Flour, Guar Hydroxypropyltrimonium Chloride, Hectorite, Hydrated Silica, Hydrogenated Potato Starch, Hydroxybutyl Methylcellulose, Hydroxyethyl Acrylate/Sodium Acryloyldimethyl Taurate Copolymer, Hydroxyethylcellulose, Hydroxyethyl Chitosan, Hydroxyethyl Ethylcellulose, Hydroxypropylcellulose, Hydroxypropyl Chitosan, Hydroxypropyl Ethylenediamine Carbomer, Hydroxypropyl Guar, Hydroxypropyl Methylcellulose, Hydroxypropyl Methylcellulose Stearoxy Ether, Hydroxystearamide MEA, Isobutylene/Sodium Maleate Copolymer, Lithium Magnesium Silicate, Lithium Magnesium Sodium Silicate, Macrocystis Pyrifera (Kelp), Magnesium Alginate, Magnesium Aluminum Silicate, Magnesium Silicate, Magnesium Trisilicate, Methoxy PEG-22/Dodecyl Glycol Copolymer, Methylcellulose, Methyl Ethylcellulose, Methyl Hydroxyethylcellulose, Microcrystalline Cellulose, Montmorillonite, Moroccan Lava Clay, Natto Gum, Nonoxynyl Hydroxyethylcellulose, Octadecene/MA Copolymer, Pectin, PEG-800, PEG-Crosspolymer, PEG-150/Decyl Alcohol/SMDI Copolymer, PEG-175 Diisostearate, PEG-190 Distearate, PEG-15 Glyceryl Tristearate, PEG-140 Glyceryl Tristearate, PEG-240/HDI Copolymer Bis-Decyltetradeceth-20 Ether, PEG-100/IPDI Copolymer, PEG-180/Laureth-50/TMMG Copolymer, PEG-10/Lauryl Dimethicone Crosspolymer, PEG-15/Lauryl Dimethicone Crosspolymer, PEG-2M, PEG-5M, PEG-7M, PEG-9M, PEG-14M, PEG-20M, PEG-23M, PEG-25M, PEG-45M, PEG-65M, PEG-90M, PEG-115M, PEG-160M, PEG-120 Methyl Glucose Trioleate, PEG-180/Octoxynol-40/TMMG Copolymer, PEG-150 Pentaerythrityl Tetrastearate, PEG-4 Rapeseedamide, PEG-150/Stearyl Alcohol/SMDI Copolymer, Polyacrylate-3, Polyacrylic Acid, Polycyclopentadiene, Polyether-1, Polyethylene/Isopropyl Maleate/MA Copolyol, Polymethacrylic Acid, Polyquaternium-52, Polyvinyl Alcohol, Potassium Alginate, Potassium Aluminum Polyacrylate, Potassium Carbomer, Potassium Carrageenan, Potassium Polyacrylate, Potato Starch Modified, PPG-14 Laureth-60 Hexyl Dicarbamate, PPG-14 Laureth-60 Isophoryl Dicarbamate, PPG-14 Palmeth-60 Hexyl Dicarbamate, Propylene Glycol Alginate, PVP/Decene Copolymer, PVP Montmorillonite, Rhizobian Gum, Ricinoleic Acid/Adipic Acid/AEEA Copolymer, Sclerotium Gum, Sodium Acrylate/Acryloyldimethyl Taurate Copolymer, Sodium Acrylates/Acrolein Copolymer, Sodium Acrylates/Acrylonitrogens Copolymer, Sodium Acrylates Copolymer, Sodium Acrylates/Vinyl Isodecanoate Crosspolymer, Sodium Acrylate/Vinyl Alcohol Copolymer, Sodium Carbomer, Sodium Carboxymethyl Chitin, Sodium Carboxymethyl Dextran, Sodium Carboxymethyl Beta-Glucan, Sodium Carboxymethyl Starch, Sodium Carrageenan, Sodium Cellulose Sulfate, Sodium Cyclodextrin Sulfate, Sodium Hydroxypropyl Starch Phosphate, Sodium Isooctylene/MA Copolymer, Sodium Magnesium Fluorosilicate, Sodium Polyacrylate, Sodium Polyacrylate Starch, Sodium Polyacryloyldimethyl Taurate, Sodium Polymethacrylate, Sodium Polystyrene Sulfonate, Sodium Silicoaluminate, Sodium Starch Octenylsuccinate, Sodium Stearoxy PG-Hydroxyethylcellulose Sulfonate, Sodium Styrene/Acrylates Copolymer, Sodium Tauride Acrylates/Acrylic Acid/Acrylonitrogens Copolymer, Solanum Tuberosum (Potato) Starch, Starch/Acrylates/Acrylamide Copolymer, Starch Hydroxypropyltrimonium Chloride, Steareth-60 Cetyl Ether, Steareth-100/PEG-136/HDI Copolymer, Sterculia Urens Gum, Synthetic Fluorphlogopite, Tamarindus Indica Seed Gum, Tapioca Starch, TEA-Alginate, TEA-Carbomer, Triticum Vulgare (Wheat) Starch, Tromethamine Acrylates/Acrylonitrogens Copolymer, Tromethamine Magnesium Aluminum Silicate, Welan Gum, Yeast Beta-Glucan, Yeast Polysaccharides, and Zea Mays (Corn) Starch.

The composition of some technically advantageous cosmetic preparations a) with a liquid carrier, in which the weight proportion of copolymers a1) and a2) in terms of the total weight of the cosmetic preparation is about 1.0 to about 10% by weight, preferably about 1.5 to about 9.0% by weight, and in particular about 2.0 to about 8.0% by weight, can be obtained from the following tables. (The quantities in percent by weight refer to the total weight of the cosmetic agent unless specified otherwise.)

| | Formula 101 | Formula 102 | Formula 103 | Formula 104 | Formula 105 |
|---|---|---|---|---|---|
| Copolymer a1)* | 0.1 to 9.9 | 0.1 to 8.9 | 0.5 to 8.0 | 0.5 to 7.5 | 1.0 to 7.0 |
| Copolymer a2)* | 0.1 to 9.9 | 0.1 to 8.9 | 0.5 to 8.0 | 0.5 to 7.5 | 1.0 to 7.0 |
| Polymeric organic | 0.05 to 8.0 | 0.05 to 8.0 | 0.1 to 5.0 | 0.1 to 5.0 | 0.1 to 5.0 |
| thickener | | | | | |
| Water and/or Ethanol | 40 to 98 | 40 to 98 | 60 to 95 | 60 to 95 | 70 to 92 |
| Optional additives | To 100 | To 100 | To 100 | To 100 | To 100 |
| | Formula 106 | Formula 107 | Formula 108 | Formula 109 | Formula 110 |
| Octylacrylamide/ | 0.1 to 9.9 | 0.1 to 8.9 | 0.5 to 8.0 | 0.5 to 7.5 | 1.0 to 7.0 |
| Acrylates/ | | | | | |
| Butylaminoethyl | | | | | |
| Methacrylate | | | | | |
| Copolymer (INCI) | | | | | |
| Copolymer a2)* | 0.1 to 9.9 | 0.1 to 8.9 | 0.5 to 8.0 | 0.5 to 7.5 | 1.0 to 7.0 |
| Polymeric organic | 0.05 to 8.0 | 0.05 to 8.0 | 0.1 to 5.0 | 0.1 to 5.0 | 0.1 to 5.0 |
| thickener | | | | | |
| Water and/or Ethanol | 40 to 98 | 40 to 98 | 60 to 95 | 60 to 95 | 70 to 92 |
| Optional additives | To 100 | To 100 | To 100 | To 100 | To 100 |
| | Formula 111 | Formula 112 | Formula 113 | Formula 114 | Formula 115 |
| Copolymer a1)* | 0.1 to 9.9 | 0.1 to 8.9 | 0.5 to 8.0 | 0.5 to 7.5 | 1.0 to 7.0 |
| Styrene/Acrylates | 0.1 to 9.9 | 0.1 to 8.9 | 0.5 to 8.0 | 0.5 to 7.5 | 1.0 to 7.0 |
| Copolymer (INCI) | | | | | |
| Polymeric organic | 0.05 to 8.0 | 0.05 to 8.0 | 0.1 to 5.0 | 0.1 to 5.0 | 0.1 to 5.0 |
| thickener | | | | | |
| Water and/or Ethanol | 40 to 98 | 40 to 98 | 60 to 95 | 60 to 95 | 70 to 92 |
| Optional additives | To 100 | To 100 | To 100 | To 100 | To 100 |
| | Formula 116 | Formula 117 | Formula 118 | Formula 119 | Formula 120 |
| Octylacrylamide/ | 0.1 to 9.9 | 0.1 to 8.9 | 0.5 to 8.0 | 0.5 to 7.5 | 1.0 to 7.0 |
| Acrylates/ | | | | | |
| Butylaminoethyl | | | | | |
| Methacrylate | | | | | |
| Copolymer (INCI) | | | | | |
| Styrene/Acrylates | 0.1 to 9.9 | 0.1 to 8.9 | 0.5 to 8.0 | 0.5 to 7.5 | 1.0 to 7.0 |
| Copolymer (INCI) | | | | | |
| Polymeric organic | 0.05 to 8.0 | 0.05 to 8.0 | 0.1 to 5.0 | 0.1 to 5.0 | 0.1 to 5.0 |
| thickener | | | | | |
| Water and/or Ethanol | 40 to 98 | 40 to 98 | 60 to 95 | 60 to 95 | 70 to 92 |
| Optional additives | To 100 | To 100 | To 100 | To 100 | To 100 |

*according to claim 1

The mousse, foam, or spray can be provided both without the addition of a propellant, for example, by means of a mechanical pump, foam, or spray device but also with use of a propellant (e.g., aerosol spray). Suitable cosmetic agents then comprise, in addition to cosmetic preparation a), further at least one propellant b).

Suitable propellants (propellant gases) are propane, propene, n-butane, isobutane, isobutene, n-pentane, pentene, isopentane, isopentene, methane, ethane, dimethyl ether, nitrogen, air, oxygen, laughing gas, 1,1,1,3-tetrafluoroethane, heptafluoro-n-propane, perfluoroethane, monochlorodifluoromethane, 1,1-difluoroethane, namely, both individually and also in combination. Hydrophilic propellant gases such as, e.g., carbon dioxide, can also be used advantageously in the context of the present disclosure, if the proportion of hydrophilic gases is selected as low and a lipophilic propellant gas (e.g., propane/butane) is present in excess. Propane, n-butane, isobutane, and mixtures of said propellant gases are particularly preferred. Preferred cosmetic agents are characterized in that the agent comprises furthermore at least one propellant b) from the group comprising propane, a mixture of propane and butane, dimethyl ether, and 1,1-difluoroethane (INCI: Hydrofluorocarbon 152a).

The further composition of some cosmetic agents, which comprise, in addition to cosmetic preparation a), further a propellant b), and in which the weight proportion of copolymers a1) and a2) in terms of the total weight of the cosmetic preparation is about 1.0 to about 10% by weight, preferably 1.5 to 9.0% by weight, and in particular about 2.0 to about 8.0% by weight, can be obtained from the following Tables 1 and 2.

Some preferred cosmetic agents with a content of cosmetic preparation a) and propellant b) are described in the following Tables 1 and 2.

Cosmetic agents with a low propellant content (e.g., mousses) are described in Table 1, and cosmetic agents with a high propellant content (e.g., sprays) in Table 2.

In Tables 1 and 2, the left column (“Formula x”) in each case refers to one of the exemplary cosmetic preparations a) of formulas 1 to 100 listed in the tables disclosed further above. The other columns two to five (“propellant”) in each case indicate the amount of propellant combined with the particular cosmetic preparation. These quantities given in “% by weight” refer to the total weight of cosmetic preparation a) of the particular “formula x” without propellant.

The quantity “4 to 12.5% by weight” in the following Table 1 therefore corresponds to the addition of propellant to cosmetic preparation a) in an amount of 4 to 12.5% by weight of the weight of cosmetic preparation a). In other words, the cosmetic preparation a) and propellant b) are present in this cosmetic agent in a weight ratio of about 100:4 to about 100:12.5 or of about 25:1 to about 8:1.

The cosmetic agents according to row 2, column 4 of the following Table 1 are, in other words, mixtures of the propellant-free cosmetic preparation a) according to formula 1 in the above table with propane/butane in a weight ratio of about 100:4 to about 100:12.5 or, stated differently, a cosmetic agent for the temporary shaping of keratinic fibers, comprising

a) a cosmetic preparation containing
a1) about 0.1 to about 9.9% by weight of at least one copolymer, made up of at least the following monomer units:
   N-tert-octylacrylamide,
   acrylic acid,
   tert-butylaminoethyl methacrylate,
a2) about 0.1 to about 9.9% by weight of at least one copolymer, made up of at least the following monomer units:
   styrene,
   acrylic acid and/or methacrylic acid,

b) propellant from the group of propane/butane,

wherein the weight proportion of copolymers a1) and a2) in terms of the total weight of the cosmetic preparation is about 1.0 to about 10% by weight and the weight ratio of cosmetic preparation a) to propellant b) is about 25:1 to about 8:1.

A first group of particularly preferred cosmetic agents of the disclosure contains, based on their total weight, about 80 to about 96% by weight of cosmetic preparation a) and about 4 to about 20% by weight of propellant, preferably about 87.5 to about 96% by weight of cosmetic preparation a) and about 4 to about 12.5% by weight of propellant b) and in particular about 92 to about 96% by weight of cosmetic preparation a) and about 4 to about 8% by weight propellant. Preferred propellants are propane/butane mixtures. Appropriate agents are suitable in particular for use as a mousse or foam:

TABLE 1

| | Propellant [% by weight] | | | |
|---|---|---|---|---|
| Formula 1 | 4 to 20 | 4 to 20 P/B* | 4 to 12.5 P/B* | 4 to 8 P/B* |
| Formula 2 | 4 to 20 | 4 to 20 P/B* | 4 to 12.5 P/B* | 4 to 8 P/B* |
| Formula 3 | 4 to 20 | 4 to 20 P/B* | 4 to 12.5 P/B* | 4 to 8 P/B* |
| Formula 4 | 4 to 20 | 4 to 20 P/B* | 4 to 12.5 P/B* | 4 to 8 P/B* |
| Formula 5 | 4 to 20 | 4 to 20 P/B* | 4 to 12.5 P/B* | 4 to 8 P/B* |
| Formula 6 | 4 to 20 | 4 to 20 P/B* | 4 to 12.5 P/B* | 4 to 8 P/B* |
| Formula 7 | 4 to 20 | 4 to 20 P/B* | 4 to 12.5 P/B* | 4 to 8 P/B* |
| Formula 8 | 4 to 20 | 4 to 20 P/B* | 4 to 12.5 P/B* | 4 to 8 P/B* |
| Formula 9 | 4 to 20 | 4 to 20 P/B* | 4 to 12.5 P/B* | 4 to 8 P/B* |
| Formula 10 | 4 to 20 | 4 to 20 P/B* | 4 to 12.5 P/B* | 4 to 8 P/B* |
| Formula 11 | 4 to 20 | 4 to 20 P/B* | 4 to 12.5 P/B* | 4 to 8 P/B* |
| Formula 12 | 4 to 20 | 4 to 20 P/B* | 4 to 12.5 P/B* | 4 to 8 P/B* |
| Formula 13 | 4 to 20 | 4 to 20 P/B* | 4 to 12.5 P/B* | 4 to 8 P/B* |
| Formula 14 | 4 to 20 | 4 to 20 P/B* | 4 to 12.5 P/B* | 4 to 8 P/B* |
| Formula 15 | 4 to 20 | 4 to 20 P/B* | 4 to 12.5 P/B* | 4 to 8 P/B* |
| Formula 16 | 4 to 20 | 4 to 20 P/B* | 4 to 12.5 P/B* | 4 to 8 P/B* |
| Formula 17 | 4 to 20 | 4 to 20 P/B* | 4 to 12.5 P/B* | 4 to 8 P/B* |
| Formula 18 | 4 to 20 | 4 to 20 P/B* | 4 to 12.5 P/B* | 4 to 8 P/B* |
| Formula 19 | 4 to 20 | 4 to 20 P/B* | 4 to 12.5 P/B* | 4 to 8 P/B* |
| Formula 20 | 4 to 20 | 4 to 20 P/B* | 4 to 12.5 P/B* | 4 to 8 P/B* |
| Formula 21 | 4 to 20 | 4 to 20 P/B* | 4 to 12.5 P/B* | 4 to 8 P/B* |
| Formula 22 | 4 to 20 | 4 to 20 P/B* | 4 to 12.5 P/B* | 4 to 8 P/B* |
| Formula 23 | 4 to 20 | 4 to 20 P/B* | 4 to 12.5 P/B* | 4 to 8 P/B* |
| Formula 24 | 4 to 20 | 4 to 20 P/B* | 4 to 12.5 P/B* | 4 to 8 P/B* |
| Formula 25 | 4 to 20 | 4 to 20 P/B* | 4 to 12.5 P/B* | 4 to 8 P/B* |
| Formula 26 | 4 to 20 | 4 to 20 P/B* | 4 to 12.5 P/B* | 4 to 8 P/B* |
| Formula 27 | 4 to 20 | 4 to 20 P/B* | 4 to 12.5 P/B* | 4 to 8 P/B* |
| Formula 28 | 4 to 20 | 4 to 20 P/B* | 4 to 12.5 P/B* | 4 to 8 P/B* |
| Formula 29 | 4 to 20 | 4 to 20 P/B* | 4 to 12.5 P/B* | 4 to 8 P/B* |
| Formula 30 | 4 to 20 | 4 to 20 P/B* | 4 to 12.5 P/B* | 4 to 8 P/B* |
| Formula 31 | 4 to 20 | 4 to 20 P/B* | 4 to 12.5 P/B* | 4 to 8 P/B* |
| Formula 32 | 4 to 20 | 4 to 20 P/B* | 4 to 12.5 P/B* | 4 to 8 P/B* |
| Formula 33 | 4 to 20 | 4 to 20 P/B* | 4 to 12.5 P/B* | 4 to 8 P/B* |
| Formula 34 | 4 to 20 | 4 to 20 P/B* | 4 to 12.5 P/B* | 4 to 8 P/B* |
| Formula 35 | 4 to 20 | 4 to 20 P/B* | 4 to 12.5 P/B* | 4 to 8 P/B* |
| Formula 36 | 4 to 20 | 4 to 20 P/B* | 4 to 12.5 P/B* | 4 to 8 P/B* |
| Formula 37 | 4 to 20 | 4 to 20 P/B* | 4 to 12.5 P/B* | 4 to 8 P/B* |
| Formula 38 | 4 to 20 | 4 to 20 P/B* | 4 to 12.5 P/B* | 4 to 8 P/B* |
| Formula 39 | 4 to 20 | 4 to 20 P/B* | 4 to 12.5 P/B* | 4 to 8 P/B* |
| Formula 40 | 4 to 20 | 4 to 20 P/B* | 4 to 12.5 P/B* | 4 to 8 P/B* |
| Formula 41 | 4 to 20 | 4 to 20 P/B* | 4 to 12.5 P/B* | 4 to 8 P/B* |
| Formula 42 | 4 to 20 | 4 to 20 P/B* | 4 to 12.5 P/B* | 4 to 8 P/B* |
| Formula 43 | 4 to 20 | 4 to 20 P/B* | 4 to 12.5 P/B* | 4 to 8 P/B* |
| Formula 44 | 4 to 20 | 4 to 20 P/B* | 4 to 12.5 P/B* | 4 to 8 P/B* |
| Formula 45 | 4 to 20 | 4 to 20 P/B* | 4 to 12.5 P/B* | 4 to 8 P/B* |
| Formula 46 | 4 to 20 | 4 to 20 P/B* | 4 to 12.5 P/B* | 4 to 8 P/B* |
| Formula 47 | 4 to 20 | 4 to 20 P/B* | 4 to 12.5 P/B* | 4 to 8 P/B* |
| Formula 48 | 4 to 20 | 4 to 20 P/B* | 4 to 12.5 P/B* | 4 to 8 P/B* |
| Formula 49 | 4 to 20 | 4 to 20 P/B* | 4 to 12.5 P/B* | 4 to 8 P/B* |
| Formula 50 | 4 to 20 | 4 to 20 P/B* | 4 to 12.5 P/B* | 4 to 8 P/B* |
| Formula 51 | 4 to 20 | 4 to 20 P/B* | 4 to 12.5 P/B* | 4 to 8 P/B* |
| Formula 52 | 4 to 20 | 4 to 20 P/B* | 4 to 12.5 P/B* | 4 to 8 P/B* |
| Formula 53 | 4 to 20 | 4 to 20 P/B* | 4 to 12.5 P/B* | 4 to 8 P/B* |
| Formula 54 | 4 to 20 | 4 to 20 P/B* | 4 to 12.5 P/B* | 4 to 8 P/B* |
| Formula 55 | 4 to 20 | 4 to 20 P/B* | 4 to 12.5 P/B* | 4 to 8 P/B* |
| Formula 56 | 4 to 20 | 4 to 20 P/B* | 4 to 12.5 P/B* | 4 to 8 P/B* |
| Formula 57 | 4 to 20 | 4 to 20 P/B* | 4 to 12.5 P/B* | 4 to 8 P/B* |
| Formula 58 | 4 to 20 | 4 to 20 P/B* | 4 to 12.5 P/B* | 4 to 8 P/B* |
| Formula 59 | 4 to 20 | 4 to 20 P/B* | 4 to 12.5 P/B* | 4 to 8 P/B* |
| Formula 60 | 4 to 20 | 4 to 20 P/B* | 4 to 12.5 P/B* | 4 to 8 P/B* |
| Formula 61 | 4 to 20 | 4 to 20 P/B* | 4 to 12.5 P/B* | 4 to 8 P/B* |
| Formula 62 | 4 to 20 | 4 to 20 P/B* | 4 to 12.5 P/B* | 4 to 8 P/B* |
| Formula 63 | 4 to 20 | 4 to 20 P/B* | 4 to 12.5 P/B* | 4 to 8 P/B* |
| Formula 64 | 4 to 20 | 4 to 20 P/B* | 4 to 12.5 P/B* | 4 to 8 P/B* |
| Formula 65 | 4 to 20 | 4 to 20 P/B* | 4 to 12.5 P/B* | 4 to 8 P/B* |
| Formula 66 | 4 to 20 | 4 to 20 P/B* | 4 to 12.5 P/B* | 4 to 8 P/B* |
| Formula 67 | 4 to 20 | 4 to 20 P/B* | 4 to 12.5 P/B* | 4 to 8 P/B* |
| Formula 68 | 4 to 20 | 4 to 20 P/B* | 4 to 12.5 P/B* | 4 to 8 P/B* |
| Formula 69 | 4 to 20 | 4 to 20 P/B* | 4 to 12.5 P/B* | 4 to 8 P/B* |
| Formula 70 | 4 to 20 | 4 to 20 P/B* | 4 to 12.5 P/B* | 4 to 8 P/B* |
| Formula 71 | 4 to 20 | 4 to 20 P/B* | 4 to 12.5 P/B* | 4 to 8 P/B* |
| Formula 72 | 4 to 20 | 4 to 20 P/B* | 4 to 12.5 P/B* | 4 to 8 P/B* |
| Formula 73 | 4 to 20 | 4 to 20 P/B* | 4 to 12.5 P/B* | 4 to 8 P/B* |
| Formula 74 | 4 to 20 | 4 to 20 P/B* | 4 to 12.5 P/B* | 4 to 8 P/B* |
| Formula 75 | 4 to 20 | 4 to 20 P/B* | 4 to 12.5 P/B* | 4 to 8 P/B* |
| Formula 76 | 4 to 20 | 4 to 20 P/B* | 4 to 12.5 P/B* | 4 to 8 P/B* |
| Formula 77 | 4 to 20 | 4 to 20 P/B* | 4 to 12.5 P/B* | 4 to 8 P/B* |
| Formula 78 | 4 to 20 | 4 to 20 P/B* | 4 to 12.5 P/B* | 4 to 8 P/B* |
| Formula 79 | 4 to 20 | 4 to 20 P/B* | 4 to 12.5 P/B* | 4 to 8 P/B* |
| Formula 80 | 4 to 20 | 4 to 20 P/B* | 4 to 12.5 P/B* | 4 to 8 P/B* |
| Formula 81 | 4 to 20 | 4 to 20 P/B* | 4 to 12.5 P/B* | 4 to 8 P/B* |
| Formula 82 | 4 to 20 | 4 to 20 P/B* | 4 to 12.5 P/B* | 4 to 8 P/B* |
| Formula 83 | 4 to 20 | 4 to 20 P/B* | 4 to 12.5 P/B* | 4 to 8 P/B* |
| Formula 84 | 4 to 20 | 4 to 20 P/B* | 4 to 12.5 P/B* | 4 to 8 P/B* |
| Formula 85 | 4 to 20 | 4 to 20 P/B* | 4 to 12.5 P/B* | 4 to 8 P/B* |
| Formula 86 | 4 to 20 | 4 to 20 P/B* | 4 to 12.5 P/B* | 4 to 8 P/B* |
| Formula 87 | 4 to 20 | 4 to 20 P/B* | 4 to 12.5 P/B* | 4 to 8 P/B* |
| Formula 88 | 4 to 20 | 4 to 20 P/B* | 4 to 12.5 P/B* | 4 to 8 P/B* |
| Formula 89 | 4 to 20 | 4 to 20 P/B* | 4 to 12.5 P/B* | 4 to 8 P/B* |
| Formula 90 | 4 to 20 | 4 to 20 P/B* | 4 to 12.5 P/B* | 4 to 8 P/B* |
| Formula 91 | 4 to 20 | 4 to 20 P/B* | 4 to 12.5 P/B* | 4 to 8 P/B* |
| Formula 92 | 4 to 20 | 4 to 20 P/B* | 4 to 12.5 P/B* | 4 to 8 P/B* |
| Formula 93 | 4 to 20 | 4 to 20 P/B* | 4 to 12.5 P/B* | 4 to 8 P/B* |
| Formula 94 | 4 to 20 | 4 to 20 P/B* | 4 to 12.5 P/B* | 4 to 8 P/B* |
| Formula 95 | 4 to 20 | 4 to 20 P/B* | 4 to 12.5 P/B* | 4 to 8 P/B* |
| Formula 96 | 4 to 20 | 4 to 20 P/B* | 4 to 12.5 P/B* | 4 to 8 P/B* |
| Formula 97 | 4 to 20 | 4 to 20 P/B* | 4 to 12.5 P/B* | 4 to 8 P/B* |
| Formula 98 | 4 to 20 | 4 to 20 P/B* | 4 to 12.5 P/B* | 4 to 8 P/B* |
| Formula 99 | 4 to 20 | 4 to 20 P/B* | 4 to 12.5 P/B* | 4 to 8 P/B* |
| Formula 100 | 4 to 20 | 4 to 20 P/B* | 4 to 12.5 P/B* | 4 to 8 P/B* |

*“P/B” corresponds to a propane/butane mixture

The quantity “50 to 200% by weight” in the following Table 2 corresponds to the addition of propellant to cosmetic preparation a) in an amount of about 50 to about 200% by weight of the weight of cosmetic preparation a). In other words, cosmetic preparation a) and propellant b) are present in this cosmetic agent in a weight ratio of about 100:50 to about 100:200 or of about 2:1 to about 1:2.

In row 4, column 3 of the following Table 2, accordingly, a mixture of the propellant-free cosmetic preparation a) according to formula 3 with a propane/butane mixture is described. The entry in row 4, column 3 accordingly describes a cosmetic agent for the temporary shaping of keratinic fibers, comprising

a) a cosmetic preparation containing
a1) about 0.5 to about 8.0% by weight of at least one copolymer, made up of at least the following monomer units:
   N-tert-octylacrylamide,
   acrylic acid,
   tert-butylaminoethyl methacrylate,
a2) about 0.5 to about 8.0% by weight of at least one copolymer, made up of at least the following monomer units:
   styrene,
   acrylic acid and/or methacrylic acid,

b) propellant from the group of propane/butane mixtures,

wherein the weight proportion of copolymers a1) and a2) in terms of the total weight of the cosmetic preparation amounts to about 1.0 to about 10% by weight and the weight ratio of cosmetic preparation a) to propellant b) is about 2:1 to about 1:2.

A second group of particularly preferred cosmetic agents of the disclosure contains, based on their total weight, about 30 to about 70% by weight of cosmetic preparation a) and about 30 to about 70% by weight of propellant b). Such agents are suitable in particular for use as a spray:

TABLE 2

| | Propellant [% by weight] | | | |
|---|---|---|---|---|
| Formula 1 | 50 to 200 | 50 to 200 P/B* | 50 to 200 DFE** | 50 to 200 DME*** |
| Formula 2 | 50 to 200 | 50 to 200 P/B* | 50 to 200 DFE** | 50 to 200 DME*** |
| Formula 3 | 50 to 200 | 50 to 200 P/B* | 50 to 200 DFE** | 50 to 200 DME*** |
| Formula 4 | 50 to 200 | 50 to 200 P/B* | 50 to 200 DFE** | 50 to 200 DME*** |
| Formula 5 | 50 to 200 | 50 to 200 P/B* | 50 to 200 DFE** | 50 to 200 DME*** |
| Formula 6 | 50 to 200 | 50 to 200 P/B* | 50 to 200 DFE** | 50 to 200 DME*** |
| Formula 7 | 50 to 200 | 50 to 200 P/B* | 50 to 200 DFE** | 50 to 200 DME*** |
| Formula 8 | 50 to 200 | 50 to 200 P/B* | 50 to 200 DFE** | 50 to 200 DME*** |
| Formula 9 | 50 to 200 | 50 to 200 P/B* | 50 to 200 DFE** | 50 to 200 DME*** |
| Formula 10 | 50 to 200 | 50 to 200 P/B* | 50 to 200 DFE** | 50 to 200 DME*** |
| Formula 11 | 50 to 200 | 50 to 200 P/B* | 50 to 200 DFE** | 50 to 200 DME*** |
| Formula 12 | 50 to 200 | 50 to 200 P/B* | 50 to 200 DFE** | 50 to 200 DME*** |
| Formula 13 | 50 to 200 | 50 to 200 P/B* | 50 to 200 DFE** | 50 to 200 DME*** |
| Formula 14 | 50 to 200 | 50 to 200 P/B* | 50 to 200 DFE** | 50 to 200 DME*** |
| Formula 15 | 50 to 200 | 50 to 200 P/B* | 50 to 200 DFE** | 50 to 200 DME*** |
| Formula 16 | 50 to 200 | 50 to 200 P/B* | 50 to 200 DFE** | 50 to 200 DME*** |
| Formula 17 | 50 to 200 | 50 to 200 P/B* | 50 to 200 DFE** | 50 to 200 DME*** |
| Formula 18 | 50 to 200 | 50 to 200 P/B* | 50 to 200 DFE** | 50 to 200 DME*** |
| Formula 19 | 50 to 200 | 50 to 200 P/B* | 50 to 200 DFE** | 50 to 200 DME*** |
| Formula 20 | 50 to 200 | 50 to 200 P/B* | 50 to 200 DFE** | 50 to 200 DME*** |
| Formula 21 | 50 to 200 | 50 to 200 P/B* | 50 to 200 DFE** | 50 to 200 DME*** |
| Formula 22 | 50 to 200 | 50 to 200 P/B* | 50 to 200 DFE** | 50 to 200 DME*** |
| Formula 23 | 50 to 200 | 50 to 200 P/B* | 50 to 200 DFE** | 50 to 200 DME*** |
| Formula 24 | 50 to 200 | 50 to 200 P/B* | 50 to 200 DFE** | 50 to 200 DME*** |
| Formula 25 | 50 to 200 | 50 to 200 P/B* | 50 to 200 DFE** | 50 to 200 DME*** |
| Formula 26 | 50 to 200 | 50 to 200 P/B* | 50 to 200 DFE** | 50 to 200 DME*** |
| Formula 27 | 50 to 200 | 50 to 200 P/B* | 50 to 200 DFE** | 50 to 200 DME*** |
| Formula 28 | 50 to 200 | 50 to 200 P/B* | 50 to 200 DFE** | 50 to 200 DME*** |
| Formula 29 | 50 to 200 | 50 to 200 P/B* | 50 to 200 DFE** | 50 to 200 DME*** |
| Formula 30 | 50 to 200 | 50 to 200 P/B* | 50 to 200 DFE** | 50 to 200 DME*** |
| Formula 31 | 50 to 200 | 50 to 200 P/B* | 50 to 200 DFE** | 50 to 200 DME*** |
| Formula 32 | 50 to 200 | 50 to 200 P/B* | 50 to 200 DFE** | 50 to 200 DME*** |
| Formula 33 | 50 to 200 | 50 to 200 P/B* | 50 to 200 DFE** | 50 to 200 DME*** |
| Formula 34 | 50 to 200 | 50 to 200 P/B* | 50 to 200 DFE** | 50 to 200 DME*** |
| Formula 35 | 50 to 200 | 50 to 200 P/B* | 50 to 200 DFE** | 50 to 200 DME*** |
| Formula 36 | 50 to 200 | 50 to 200 P/B* | 50 to 200 DFE** | 50 to 200 DME*** |
| Formula 37 | 50 to 200 | 50 to 200 P/B* | 50 to 200 DFE** | 50 to 200 DME*** |
| Formula 38 | 50 to 200 | 50 to 200 P/B* | 50 to 200 DFE** | 50 to 200 DME*** |
| Formula 39 | 50 to 200 | 50 to 200 P/B* | 50 to 200 DFE** | 50 to 200 DME*** |
| Formula 40 | 50 to 200 | 50 to 200 P/B* | 50 to 200 DFE** | 50 to 200 DME*** |
| Formula 41 | 50 to 200 | 50 to 200 P/B* | 50 to 200 DFE** | 50 to 200 DME*** |
| Formula 42 | 50 to 200 | 50 to 200 P/B* | 50 to 200 DFE** | 50 to 200 DME*** |
| Formula 43 | 50 to 200 | 50 to 200 P/B* | 50 to 200 DFE** | 50 to 200 DME*** |
| Formula 44 | 50 to 200 | 50 to 200 P/B* | 50 to 200 DFE** | 50 to 200 DME*** |
| Formula 45 | 50 to 200 | 50 to 200 P/B* | 50 to 200 DFE** | 50 to 200 DME*** |
| Formula 46 | 50 to 200 | 50 to 200 P/B* | 50 to 200 DFE** | 50 to 200 DME*** |
| Formula 47 | 50 to 200 | 50 to 200 P/B* | 50 to 200 DFE** | 50 to 200 DME*** |
| Formula 48 | 50 to 200 | 50 to 200 P/B* | 50 to 200 DFE** | 50 to 200 DME*** |
| Formula 49 | 50 to 200 | 50 to 200 P/B* | 50 to 200 DFE** | 50 to 200 DME*** |
| Formula 50 | 50 to 200 | 50 to 200 P/B* | 50 to 200 DFE** | 50 to 200 DME*** |
| Formula 51 | 50 to 200 | 50 to 200 P/B* | 50 to 200 DFE** | 50 to 200 DME*** |
| Formula 52 | 50 to 200 | 50 to 200 P/B* | 50 to 200 DFE** | 50 to 200 DME*** |
| Formula 53 | 50 to 200 | 50 to 200 P/B* | 50 to 200 DFE** | 50 to 200 DME*** |
| Formula 54 | 50 to 200 | 50 to 200 P/B* | 50 to 200 DFE** | 50 to 200 DME*** |
| Formula 55 | 50 to 200 | 50 to 200 P/B* | 50 to 200 DFE** | 50 to 200 DME*** |
| Formula 56 | 50 to 200 | 50 to 200 P/B* | 50 to 200 DFE** | 50 to 200 DME*** |
| Formula 57 | 50 to 200 | 50 to 200 P/B* | 50 to 200 DFE** | 50 to 200 DME*** |
| Formula 58 | 50 to 200 | 50 to 200 P/B* | 50 to 200 DFE** | 50 to 200 DME*** |
| Formula 59 | 50 to 200 | 50 to 200 P/B* | 50 to 200 DFE** | 50 to 200 DME*** |
| Formula 60 | 50 to 200 | 50 to 200 P/B* | 50 to 200 DFE** | 50 to 200 DME*** |
| Formula 61 | 50 to 200 | 50 to 200 P/B* | 50 to 200 DFE** | 50 to 200 DME*** |
| Formula 62 | 50 to 200 | 50 to 200 P/B* | 50 to 200 DFE** | 50 to 200 DME*** |
| Formula 63 | 50 to 200 | 50 to 200 P/B* | 50 to 200 DFE** | 50 to 200 DME*** |
| Formula 64 | 50 to 200 | 50 to 200 P/B* | 50 to 200 DFE** | 50 to 200 DME*** |
| Formula 65 | 50 to 200 | 50 to 200 P/B* | 50 to 200 DFE** | 50 to 200 DME*** |
| Formula 66 | 50 to 200 | 50 to 200 P/B* | 50 to 200 DFE** | 50 to 200 DME*** |
| Formula 67 | 50 to 200 | 50 to 200 P/B* | 50 to 200 DFE** | 50 to 200 DME*** |
| Formula 68 | 50 to 200 | 50 to 200 P/B* | 50 to 200 DFE** | 50 to 200 DME*** |
| Formula 69 | 50 to 200 | 50 to 200 P/B* | 50 to 200 DFE** | 50 to 200 DME*** |
| Formula 70 | 50 to 200 | 50 to 200 P/B* | 50 to 200 DFE** | 50 to 200 DME*** |
| Formula 71 | 50 to 200 | 50 to 200 P/B* | 50 to 200 DFE** | 50 to 200 DME*** |
| Formula 72 | 50 to 200 | 50 to 200 P/B* | 50 to 200 DFE** | 50 to 200 DME*** |
| Formula 73 | 50 to 200 | 50 to 200 P/B* | 50 to 200 DFE** | 50 to 200 DME*** |
| Formula 74 | 50 to 200 | 50 to 200 P/B* | 50 to 200 DFE** | 50 to 200 DME*** |
| Formula 75 | 50 to 200 | 50 to 200 P/B* | 50 to 200 DFE** | 50 to 200 DME*** |
| Formula 76 | 50 to 200 | 50 to 200 P/B* | 50 to 200 DFE** | 50 to 200 DME*** |
| Formula 77 | 50 to 200 | 50 to 200 P/B* | 50 to 200 DFE** | 50 to 200 DME*** |
| Formula 78 | 50 to 200 | 50 to 200 P/B* | 50 to 200 DFE** | 50 to 200 DME*** |
| Formula 79 | 50 to 200 | 50 to 200 P/B* | 50 to 200 DFE** | 50 to 200 DME*** |
| Formula 80 | 50 to 200 | 50 to 200 P/B* | 50 to 200 DFE** | 50 to 200 DME*** |
| Formula 81 | 50 to 200 | 50 to 200 P/B* | 50 to 200 DFE** | 50 to 200 DME*** |
| Formula 82 | 50 to 200 | 50 to 200 P/B* | 50 to 200 DFE** | 50 to 200 DME*** |
| Formula 83 | 50 to 200 | 50 to 200 P/B* | 50 to 200 DFE** | 50 to 200 DME*** |
| Formula 84 | 50 to 200 | 50 to 200 P/B* | 50 to 200 DFE** | 50 to 200 DME*** |
| Formula 85 | 50 to 200 | 50 to 200 P/B* | 50 to 200 DFE** | 50 to 200 DME*** |
| Formula 86 | 50 to 200 | 50 to 200 P/B* | 50 to 200 DFE** | 50 to 200 DME*** |
| Formula 87 | 50 to 200 | 50 to 200 P/B* | 50 to 200 DFE** | 50 to 200 DME*** |
| Formula 88 | 50 to 200 | 50 to 200 P/B* | 50 to 200 DFE** | 50 to 200 DME*** |
| Formula 89 | 50 to 200 | 50 to 200 P/B* | 50 to 200 DFE** | 50 to 200 DME*** |
| Formula 90 | 50 to 200 | 50 to 200 P/B* | 50 to 200 DFE** | 50 to 200 DME*** |
| Formula 91 | 50 to 200 | 50 to 200 P/B* | 50 to 200 DFE** | 50 to 200 DME*** |
| Formula 92 | 50 to 200 | 50 to 200 P/B* | 50 to 200 DFE** | 50 to 200 DME*** |
| Formula 93 | 50 to 200 | 50 to 200 P/B* | 50 to 200 DFE** | 50 to 200 DME*** |
| Formula 94 | 50 to 200 | 50 to 200 P/B* | 50 to 200 DFE** | 50 to 200 DME*** |
| Formula 95 | 50 to 200 | 50 to 200 P/B* | 50 to 200 DFE** | 50 to 200 DME*** |
| Formula 96 | 50 to 200 | 50 to 200 P/B* | 50 to 200 DFE** | 50 to 200 DME*** |
| Formula 97 | 50 to 200 | 50 to 200 P/B* | 50 to 200 DFE** | 50 to 200 DME*** |
| Formula 98 | 50 to 200 | 50 to 200 P/B* | 50 to 200 DFE** | 50 to 200 DME*** |
| Formula 99 | 50 to 200 | 50 to 200 P/B* | 50 to 200 DFE** | 50 to 200 DME*** |
| Formula 100 | 50 to 200 | 50 to 200 P/B* | 50 to 200 DFE** | 50 to 200 DME*** |

*“P/B” corresponds to a propane/butane mixture
**“DFE” corresponds to 1,1-difluoroethane
***“DME” corresponds to dimethyl ether

Vessels made of metal (aluminum, tin plate, tin), protected or non-splintering plastic or of glass that is externally coated with plastic may be used as compressed-gas containers for aerosol applications; pressure resistance and breaking strength, corrosion resistance, ease of filling, as well as aesthetic aspects, handling, printability, etc., play a role in their selection. Special protective interior coatings assure corrosion resistance to the cosmetic agent a).

If the agents of the disclosure are to be sprayed onto the hair, these agents are advantageously provided with a dispensing device and a spray valve. The resulting cosmetic products comprise accordingly a cosmetic agent of the disclosure and a dispensing device with a spray valve.

In a preferred embodiment of the disclosure, the valve has a valve cone covered with a coating or a polymeric plastic A and just such a flexible element with a reset function, which after the operation ends resets the valve to the closed position (=neutral position of the valve). Corresponding cosmetic products in which the aerosol dispensing device comprises a valve, which has a valve cone and/or a flexible element with a reset function, which is/are covered with a coating or a polymeric plastic A, are preferred as contemplated herein.

In another preferred embodiment of the disclosure, the valve has a flexible element with a reset function and/or a valve cone made of at least one plastic B, preferably an elastomeric plastic. Here as well, cosmetic products of the disclosure, in which the valve has a flexible element with a reset function and/or a valve cone made of at least one plastic B, are preferred, preferred plastics B being elastomeric plastics. Particularly preferred elastomeric plastics are selected from Buna, particularly Buna N, Buna 421, Buna 1602, and Buna KA 6712, neoprene, butyl, and chlorobutyl.

In another preferred embodiment of the disclosure, the flexible element with a reset function can be formed as a spiral spring or helical compression spring. In another preferred embodiment of the disclosure, the flexible element with the reset function can be formed integrally with the valve cone and have flexible legs.

As initially stated, the previously described cosmetic agents are characterized by particular hair-cosmetic properties, in particular advantageous properties during temporary hair shaping. A second subject of the present application therefore is the use of an agent of the disclosure for the temporary shaping of keratin-containing fibers, in particular human hair.

A third subject of the present application is a method for the temporary shaping of keratin-containing fibers, in particular human hair, in which the keratinic fibers are acted upon by a cosmetic agent of the disclosure and are fixed temporarily in their shape.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

1. A cosmetic agent for the temporary shaping of keratinic fibers, comprising:
a cosmetic preparation containing:
at least one copolymer a1), made up of at least the following monomer units;
N-tert-octylacrylamide,
acrylic acid, and
tert-butylaminoethyl methacrylate; and
at least one copolymer a2), made up of at least the following monomer units;
styrene, and
at least one of acrylic acid or methacrylic acid;
wherein the weight proportion of copolymers a1) and a2) in terms of the total weight of the cosmetic preparation is about 1.0 to about 10% by weight.

2. The cosmetic agent according to claim 1, wherein the weight proportion of copolymers a1) and a2) in terms of the total weight of the cosmetic preparation is about 1.5 to about 9.0% by weight.

3. The cosmetic agent according to claim 1, wherein the preparation contains, based on its total weight, about 0.1 to about 9.9% by weight of copolymer a1).

4. The cosmetic agent according to claim 1, wherein the preparation contains, based on its total weight, about 0.1 to about 9.9% by weight of copolymer a2).

5. The cosmetic agent according to claim 1, wherein the weight ratio of copolymer a1) to copolymer a2) is from about 1:7 to about 7:1.

6. The cosmetic agent according to claim 1, wherein the agent comprises, furthermore, at least one propellant.

7. The cosmetic agent according to claim 1, comprising, based on its total weight:
about 30 to about 70% by weight of the cosmetic preparation; and
about 30 to about 70% by weight of propellant.

8. A cosmetic product comprising:
a cosmetic agent according to claim 1; and
a dispensing device with a spray valve.

9. (canceled)

10. (canceled)

11. The cosmetic agent according to claim 1, wherein the weight proportion of copolymers a1) and a2) in terms of the total weight of the cosmetic preparation is about 2.0 and about 8.0% by weight.

12. The cosmetic agent according to claim 1, wherein the preparation contains, based on its total weight, about 0.5 to about 8.5% by weight of copolymer a1).

13. The cosmetic agent according to claim 1, wherein the preparation contains, based on its total weight, about 1.0 to about 7.0% by weight of copolymer a1).

14. The cosmetic agent according to claim 1, wherein the preparation contains, based on its total weight, about 0.5 to about 8.5% by weight of copolymer a2).

15. The cosmetic agent according to claim 1, wherein the preparation contains, based on its total weight, about 1.0 to about 7.0% by weight of copolymer a2).

16. The cosmetic agent according to claim 1, wherein the weight ratio of copolymer a1) to copolymer a2) is from about 1:5 to about 5:1.

17. The cosmetic agent according to claim 1, wherein the weight ratio of copolymer a1) to copolymer a2) is from about 1:3 to about 3:1.

18. The cosmetic agent according to claim 1, wherein copolymer a1) consists of at least 90% by weight of the monomers: N-tert-octylacrylamide, acrylic acid, tert-butylaminoethyl methacrylate, methyl methacrylate, and hydroxypropyl methacrylate.

19. The cosmetic agent according to claim 1, wherein the at least one copolymer a2), based on its total weight, consists of at least 90% by weight of the monomers: styrene, at least one of acrylic acid or methacrylic acid, and at least one of acrylic acid esters or methacrylic acid esters.

20. The cosmetic agent according to claim 1, wherein:
copolymer a1) has the INCI name Octylacrylamide/Acrylates/Butylaminoethyl Methacrylate Copolymer; and
copolymer a2) has the INCI name Styrene/Acrylates Copolymer.

21. A cosmetic agent for the temporary shaping of keratinic fibers, comprising:
a cosmetic preparation containing:
at least one copolymer a1), made up of at least the following monomer units;
N-tert-octylacrylamide,
acrylic acid, and
tert-butylaminoethyl methacrylate;
at least one copolymer a2), made up of at least the following monomer units;
styrene, and
at least one of acrylic acid or methacrylic acid;
at least one propellant from the group propane, mixture of propane and butane, dimethyl ether and 1,1-difluoroethane;
ethanol; and
water;
wherein the weight proportion of copolymers a1) and a2) in terms of the total weight of the cosmetic preparation is about 1.0 to about 10% by weight.

22. A method for the temporary shaping of keratinic fibers, the method comprising the steps of:
acting on keratinic fibers by a cosmetic agent comprising:
at least one copolymer a1), made up of at least the following monomer units;
N-tert-octylacrylamide,
acrylic acid, and
tert-butylaminoethyl methacrylate; and
at least one copolymer a2), made up of at least the following monomer units;
styrene, and
at least one of acrylic acid or methacrylic acid;
wherein the weight proportion of copolymers a1) and a2) in terms of the total weight of the cosmetic agent is about 1.0 to about 10% by weight; and
temporarily fixing the keratinic fibers into shape.
